(19) United States
(12) Patent Application Publication
(10) Pub. No.: US 2011/0189283 A1
(43) Pub. Date: Aug. 4, 2011

(54) APPETISING MEDICAMENTS FOR ORAL ADMINISTRATION IN SOLID FORM FOR PREVENTION AND/OR TREATMENT OF CARDIAC INSUFFICIENCY IN ANIMALS

(75) Inventors: Guy Derrieu, Cagnes sur Mer (FR); David Corvaisier, Valbonne (FR)

(73) Assignee: VIRBAC, Carros (FR)

(21) Appl. No.: 13/055,357

(22) PCT Filed: Jul. 23, 2009
(86) PCT No.: PCT/FR2009/000917

§ 371 Date: Apr. 21, 2011

(30) Foreign Application Priority Data
Jul. 23, 2008 (FR) 0804184

(51) Int. Cl. A61K 9/38; A61K 31/501; A61P 9/00; A61K 9/28; A61J 3/06; A61K 9/30

(52) U.S. Cl. 424/477; 514/252.06; 427/2.14; 427/2.22; 424/475

(57) ABSTRACT

The invention relates to the use of a coating composition for application to a solid veterinary pharmaceutical composition for oral administration for the prevention and/or treatment of cardiac insufficiency in animals, by a method of film coating comprising a powder appetizing material, a binder and a solvent. The invention further relates to a coating method by film coating of solid veterinary pharmaceutical compositions for oral administration for the prevention and/or treatment of cardiac insufficiency in animals and appetizing medicaments for animals comprising a solid veterinary pharmaceutical composition for oral administration for the prevention and/or treatment of cardiac insufficiency in animals made from pimobendan and an appetizing coating arranged around said composition comprising an appetizing material and a binder.

The present invention relates to the field of the preparation of veterinary medicaments and is targeted at improving the oral taking of a solid veterinary pharmaceutical composition for oral administration for the prevention and/or treatment of cardiac insufficiency in animals. The present invention is targeted more particularly at improving the oral taking of pimobendan, 4,5-dihydro-6-[2-(4-methoxyphenyl)-1H-benzimidazol-5-yl]-5-methyl-3(2H)-pyridazinone, formulated in a solid form, by carnivores.

Pimobendan, described in European patent EP 0 008 391, is a substance having cardiotonic, hypotensive and antithrombotic activities. It is the reference substance in the treatment of cardiac insufficiency.

The present invention is an appetizing solid veterinary pharmaceutical composition for oral administration for the prevention and/or treatment of cardiac insufficiency in animals which facilitates the spontaneous oral taking by and the administration of medicinal substances to domestic carnivores and in particular dogs and cats. A change is currently taking place in the care given to pets and the oral route is becoming a favored route for the administration of medicaments by the health professional or by the owner.

This is because there are disadvantages to the conventional parenteral routes (intramuscular, subcutaneous, intradermal or intravenous) for the administration of medicaments. The intramuscular and subcutaneous routes can, for example, cause hematomas or abscesses. With regard to the intravenous route, it often requires the intervention of a specialist (veterinarian). These parenteral administration routes also require the support of the animals. Furthermore, some active principles are difficult to formulate in parenteral pharmaceutical dosage forms. Finally, some active principles will only exert their therapeutic effect on the animal if they arrive directly in the digestive tract.

The pharmaceutical dosage formulations suited to the administration of medicaments by the oral route or per os are generally provided in the liquid form (such as syrups, solutions or suspensions to be taken orally, drops, and the like), in the semisolid form (such as slurries for oral administration) or in the solid form. The solid forms commonly used to administer medicaments to pets, in particular cats and dogs, are presented in various formulations which are different in nature and which are obtained by different processes. Singled out are, for example, hard gelatin capsules, soft capsules, tablets, capsules, compressed tablets, chewing gums, pills, pastilles or lozenges.

The hard gelatin capsule form is particularly preferred. In this form, the active substance occurs in an environment particularly favorable to its stability. This is because it is thus possible to limit all kinds of deterioration due to contact with other substances, such as excipients, fillers or appetizing materials, which occur during the preparation of other solid forms for oral administration, such as compressed tablets, pills, pastilles or lozenges, the manufacture of which employs high physical and mechanical stresses.

It is found that the observance of the treatments administered by the oral route (that is to say, abiding by the instructions and orders of the health professional relating to the taking of the medicaments) is not always appropriately followed, due to the difficulty in administering the full treatments to the animals. This is because the administration to the animals of medicaments in the solid pharmaceutical dosage form by the oral route is often difficult due to the bad taste of some active substances and of some excipients of which the medicament is composed and to the highly developed sense of smell and taste of the animals. Furthermore, the oral administration of a medicament involves, in the majority of cases, the use of variable doses according to the individuals and pathologies under consideration and sometimes requires being split up and repeated over time. It has been observed, particularly in dogs and cats, that the main reason which makes the observance of an oral treatment very difficult, indeed even impossible, is the lack of appetence aroused by the medicament.

The appetence for a medicament is defined as the psychological state corresponding to a desire to absorb the medicament in response to the perception of the organoleptic features of the latter. The ability to arouse the appetence is known as appetence arousability. It corresponds to the combination of the features of a medicament which determine the attraction which it will have over normally nourished animals. The appetence arousability of a medicament is a major contributor to the refusal or to the acceptance by the animal of the spontaneous taking of the treatment and of the repetition of the taking over periods which are sometimes lengthy. In the context of some treatments, the taking of the treatment can be daily and for life.

The appetence arousability of a medicament administered by the oral route results in the acceptance and in the voluntary ingestion by the animals. This appetence arousability can be measured in a general test of appetence which takes into account various parameters of the medicament, such as the spontaneous taking thereof from the hand or from the ground, or also the consumption thereof, even if there are several medicaments to be taken all at once or at regular intervals by the animal.

In the prior art, numerous solutions have been developed for facilitating the absorption of the medicaments by the animal:

1. A first option consists in masking the unpleasant taste and/or the unpleasant smell of the active principle or principles by encapsulation of or by coating these active principles.

Thus, in patent application EP 0 997 143, the problem of bitterness of the active principles is solved by encapsulating them in compositions which mask the taste, such as blends of cellulose acetate or of cellulose acetate butyrate or of polyvinylpyrrolidone (PVP) or of hydroxypropyl cellulose (HPC).

Patent application EP 1 490 037 provides for the preparation of a medicament in which the unpleasant taste of the active principle is masked; the preparation consists in coating the active principle, the taste of which it is desired to mask, around granules acting as support; the layer of active principle is subsequently covered with a protective layer produced with a physiologically acceptable polymer matrix (such as cellulose, starch, sucrose, lactose or other types of sugar) which prevents the active principle from being directly in contact with the gustatory cells of the animal. The granules thus prepared can subsequently be mixed with an appetizing substance and the mixture is then compressed in the lozenge form, for example.

However, these solutions require numerous encapsulating or coating stages. Furthermore, the preparation of the medicinal products in the solid pharmaceutical dosage form requires the active principles to be directly in contact with other substances, such as water or organic solvents, which make it possible either to coat the active principles with polymers or to obtain active principles in solution, which solution is used for the coating of supports by these same active principles. Manufacturing is subsequently continued, generally by compression; in point of fact, some active principles are very delicate and do not withstand the chemical and physical stresses employed during the preparation of the medicinal products with, as final stage, the compression.

International application WO 01/15547 describes a formulation of active agents which are poorly accepted by the animal for veterinary use. Here again, the principle proposed is to encapsulate the active agent or agents, incorporated beforehand in a matrix, in order to mask the taste thereof. The matrix incorporating the active agents is covered with a layer having a neutral taste which comprises xanthan, polyvinylpyrrolidone and sodium lauryl sulfate. No appetizing substance is used. Thus formulated, the active agent or agents are no longer noticed by the animal.

However, the process described in this document requires in particular an extrusion stage which is capable of damaging the delicate active principles.

2. According to another option, the active principles are mixed in a matrix comprising an appetizing substance in order to mask their taste.

Thus, Australian patent application AU2001279664 describes a starch-based extruded product comprising an active principle and a specific aromatizing agent which is capable of being administered orally to an animal.

However, the extrusion can present problems of decomposition of some active principles, for which it is preferable to avoid it.

Furthermore, an aromatizing agent is an odorous principle of synthetic or natural origin which will be noticed only by the sense of smell. In particular, it will not produce a feeling on the taste organ and thus will not have flavor. Consequently, the quality of an aromatizing agent as material capable of increasing the appetence arousability is limited.

Patent application EP 0 320 320 describes a compressed tablet for a domestic animal, characterized in that it is composed of at least one core comprising one or more active principles completely incorporated in a matrix appetent for the animal. The appetence arousability of the compressed tablet is conferred by agents, such as liver powder or beer yeast, which are constituents of the matrix, which furthermore comprises tableting excipients and/or lubricating agents, the role of which is that of facilitating the manufacture of the compressed tablet.

However, the preparation of these compressed tablets requires a tableting stage for incorporating the core comprising the active principles, which constitutes a disadvantage in that it is capable of damaging the active principle or principles. Furthermore, the active principle or principles are found to be in intimate contact with the appetent constituents of the matrix which are not necessarily compatible in order to provide both the active principles and the other constituents of the matrix, in particular the appetent constituents of the matrix, with good stability. Furthermore, this double compression technology (core, on the one hand, and appetent matrix, on the other hand) is difficult to employ; this is because it is difficult to properly center the core in the appetent matrix. This technology cannot be applied to small compressed tablets, otherwise suitable for small breeds of dogs or cats.

Patent application EP 0 725 570 of the Applicant Company describes a composition attractive for the animal in the solid form, with a sufficiently large volume not to be swallowed but chewed by the target species, comprising from 3 to 20% of a water-insoluble polymer, from 5 to 45% by weight of an appetizing substance and from 35 to 60% by weight of a lipid substance obtained by melting the lipid substances in the solid form at a temperature lower than that of the melting point of the polymer(s) and mixing the polymer(s) with the other components at the same temperature. The composition can comprise up to 50% by weight of bioactive substance and can be shaped into blocks. It is a formulation which exhibits a high mechanical strength for medicaments capable of being scattered in the environment in order to treat, for example, wild animals. The appetizing substance is distributed uniformly in the resulting pharmaceutical dosage form.

The preparation of such compositions requires heating between 40 and 80° C. in order to bring about the melting of some constituents. This heating can, however, constitute a major disadvantage for the stability of some active principles.

The limits of the formulations in which the aromatizing agents and/or the appetizing materials and the active principles are mixed and thus in contact are:
that they can lead to incompatibilities which result in the conversion or decomposition of the active principle or principles or of the aromatizing agents and of the appetizing materials. Furthermore, even present in a high amount, the appetizing materials do not always mask the smell or taste unpleasant to the animal, which has highly developed senses of smell and taste; this is all the truer if the formulation comprises liquid or solid lipids which are known to intensify flavors;
that the homogeneous distribution of the appetizing material inside the compositions renders the majority of the appetizing material unavailable to exert the maximum appetence arousability before the animal has the formulation in its mouth and begins to masticate it in order to experience the taste thereof, if appropriate; and
that these formulations do not always make it possible to protect the active principle from the environmental conditions, such as humidity and temperature, and to maintain complete stability over time.

3. Yet another option for facilitating the oral administration of active principles is to coat the medicament in appetizing materials.

The following patent applications: FR 2 715 803, U.S. Pat. No. 5,853,757, U.S. Pat. No. 6,143,316, U.S. Pat. No. 5,792,470, U.S. Pat. No. 5,674,515, EP 0 574 301, U.S. Pat. No. 4,857,333, DE 198 53 729, WO 03/030863, WO 2004/043427 and WO 2007/090987, provide for lures produced with appetizing materials into which a medicament in the solid form can be introduced at the time of use in order to administer it orally to the animal. These systems exhibit the advantage of being suitable for numerous solid forms of veterinary medicaments for the oral route.

According to a similar principle, the application WO 89/12442 describes a fish veterinary composition support comprising an external layer impermeable to water and to the active principles; this external layer surrounds at least one chamber comprising at least one active principle and masks the taste of said active principle. The external layer is composed of a plant or animal material, preferably, of fish meal, and/or of an aqueous extract of sea material, and optionally of a taste enhancer and/or of a binder. An important characteristic of this fish veterinary composition support is its impermeable nature, in particular towards water, which is obtained by the use of a binder derived from starch and by the presence of the aqueous extract of sea material, which acts as a natural adhesive.

The common disadvantage of these lures is that their use requires the prior operation of introducing the medicament into the lure, which can put off some users and can also become a nuisance when a large number of animals have to be treated. Furthermore, their large volume (necessarily bigger than the medicament) requires a large amount of material and the manufacture of them has to be suited to their complex shape; thus, these lures often prove to be expensive.

The patent application EP 0 725 627 of the Applicant Company describes a pharmaceutical dosage form for the oral administration of bioactive (biological, chemical or medicinal) substances of the bait type attractive for the animal in the solid form, with a sufficiently large volume not to be swallowed but chewed by the target species. It is a twin-compartment pharmaceutical dosage form comprising:

(i) as first compartment, a porous solid central core comprising one or more bioactive substances; this core has a specific composition suitable for solidifying by lyophilization and it exhibits the characteristic of dissolving or disintegrating rapidly in the saliva; thus, when the animal chews this medicament, the fragments generated by the core will adhere to the tissues of the oral cavity of the animal; and

(ii) a second compartment which is a hydrophobic external layer comprising from 3 to 30% of a natural or synthetic appetizing substance of the meat meal or fish meal type, from 40 to 93% of a lipid substance and from 4 to 30% of polymer. The composition of the second compartment has the specific feature of comprising lipids in order to render it hydrophobic and to protect the core against water; it also comprises polymers, which are a structural filler having the role of strengthening the lipid layer, of facilitating the handling of the bait and of modifying the melting point of the composition; this layer is thus hydrophobic and appetent and of controlled thickness. However, the preparation of such a formulation imposes mechanical and thermal constraints which are not favorable to the stability of some active principles.

In the “hard gelatin capsules” or “soft capsules” presentations, forms which are usually rejected by the animal, it is possible to add aromatizing agents to the wall of the capsules, including gelatin capsules. However, the content of aromatizing agents is necessarily low due to the constituents of the wall and its low weight with respect to the total weight of the full capsules, including gelatin capsules. Thus, the presence of aromatizing agent is often insufficient to exert a desired appetence arousability and to promote the taking, all the more so as the aromatizing agent is noticed only by the sense of smell, it does not produce a sensation on the taste organ and thus does not have flavor. Furthermore, the amount of aromatizing agent, in the liquid form, introduced can only be low (generally less than 2 percent) and has to be compatible with the nature of the component of the wall, which is gelatin in the majority of cases.

Patent application EP 0 025 226 describes compositions for coating various solid food or pharmaceutical forms for human use; said compositions are composed of sucrose, of at least one other sugar, such as lactose, and of water and can additionally comprise colorants, flavoring agents, fragrances and adjuvants. These compositions facilitate the use of a process for coating by sugar-coating by virtue of a composition formed of sugars which renders them less liable to crystallize during the process. The coating made of sugar, as in all sugar-coated tablets, confers a taste pleasant to man. However, this type of formulation is not suitable for animals, purified sugars not being a material appetizing for animals; in addition, they do not give off a smell necessary to bring about appetence arousability in animals. Finally, the “sugar-coated” form is poorly accepted by animals. This form, which is extremely hard and which has a very smooth surface, is difficult for animals to prehend.

In addition, such a coating composition is not compatible with possible addition of material appetizing for animals in the pulverulent form as:
sugar syrup with a high content of appetizing material is liable to bring about poor flow in the sugar-coating nozzles during the process for the preparation of the sugar-coated tablets;
the crystallization of the sugar during the evaporation of the solvent during the preparation of the sugar-coated tablets will have the effect of trapping the appetizing material, thus greatly reducing the attraction of the appetizing material for the animal.

There thus exists a need to improve the appetence arousability of solid medicaments administered by the oral route to animals.

This need is particularly important for the medicaments which require a frequent and repeated administration. This is in particular the case for the medicaments intended to prevent or treat chronic pathologies. They may, for example, have to be administered daily, one or more times per day. Furthermore, if the treatment protocol provides for the administration of the oral medicament to have to be offset with respect to the taking of food, then it is even more important for the medicament to be appetizing as it will be impossible to conceal the composition in the food in order to facilitate the taking thereof.

An important category of medicaments intended to prevent or treat chronic diseases is that of the medicaments used in the case of cardiac insufficiency. These pathologies are of considerable importance in veterinary medicine and there exists a constant need to improve the treatments.

The Applicant Company set itself the aim of overcoming the disadvantages of the prior art and of developing a composition and a process which make it possible to confer, on medicaments for oral administration in solid form for the prevention and/or treatment of cardiac insufficiency in animals, a better appetence arousability than the known compositions and techniques. It has in particular applied itself to developing a veterinary composition for oral administration which is simple and economical, which can be used whatever the solid pharmaceutical dosage form employed and the manufacture of which can be easily carried out on the industrial scale. In particular, the treatment for improving the appetence arousability of the medicament must be able to be applied at the end of the line for the manufacture of said medicament while ensuring the chemical and physical stability of the latter.

In the studies which led to the development of the coating composition according to the invention, the Applicant Company found that the addition of a substance appetizing for the target animal, which exists in the pulverulent form in the starting material state, in an amount concentrated at the surface of a solid composition administered by the oral route, promoted, on the one hand, the appetence of the animal and, on the other hand, prompted the animal to consume said solid composition. The substances which confer appetence arousability on a solid composition administered by the oral route depend on the tastes and smells perceived and assessed by the target animal.

The invention uses this observation as a starting point to provide an appetizing medicament for oral administration for the prevention and/or treatment of cardiac insufficiency in animals which is very well accepted by animals and which is rapidly absorbed, whether given occasionally or repeatedly.

The compounds of the following therapeutic categories are conventionally used in the treatment of cardiac insufficiency: angiotensin converting enzyme inhibitors, phosphodiesterase inhibitors, aldosterone antagonists and, finally, diuretics. Other important therapeutic categories are also used in the treatment of cardiac insufficiency but do not necessarily have applications approved by the administrative authority for use in veterinary medicine. Mention may be made, by way of examples, of cardiotonic glycosides, nitro derivatives or sympathomimetics.

As regards more specifically Angiotensin Converting Enzyme Inhibitors (ACEIs), patent medicines are found based on the following therapeutic agents: benazepril, enalapril, imidapril and ramipril. ACEIs are indicated in the case of an emerging (class II according to the NYHA classification), moderate (class III) or severe (class IV) symptomatic cardiac insufficiency, most frequently as a result of valve insufficiency (mitral or tricuspid) or dilated cardiomyopathy. They inhibit the converting enzyme, which is responsible for the conversion of angiotensin I to angiotensin II, which is the only active form. The converting enzyme is also involved in the decomposition of bradykinin. As a result of the vasoconstrictive effects of angiotensin II, ACEIs bring about a vasodilating arterial and venous effect. ACEIs also inhibit the retention of water and the vasoconstriction brought about by vasopressin and also the retention of water and of sodium and the removal of potassium controlled by aldosterone, two hormones stimulated by angiotensin II. These hypotensive effects are achieved at the expense of an activation of the renin and of a plasma accumulation of angiotensin I. The ACEIs can be combined with other conventional treatments for cardiac insufficiency. Thus, when different treatments requiring oral taking in solid form are combined, it is important for the animal to readily accept the taking of multiple oral forms.

As regards the phosphodiesterase inhibitors, patent medicines are found based on pimobendan. Pimobendan is indicated in the treatment of cardiac insufficiency as a result of cardiomyopathy or of valve insufficiency (mitral and/or tricuspid). Phosphodiesterase type III inhibitors exert a positive inotropic effect and have vasodilating properties exerted at the arterial and venous level, thus resulting in a reduction in preload and afterload. The inhibition of the decomposition of cyclic AMP and the increase in the calcium sensitivity of the cardiac fibers contribute to these effects. Some of these molecules have a very high affinity for the cardiovascular system due to their selectivity for a specific phosphodiesterase isoenzyme. The positive inotropic effects of pimobendan are observed for 8 to hours after oral administration. After oral administration, the bioavailability of the pimobendan is from 60 to 63%. This bioavailability is considerably reduced when the active principle is administered during or shortly after feeding. Thus, it is recommended not to feed the dog in the hour which follows the administration of the patent medicine. Pimobendan can be used in the dog simultaneously with other conventional treatments for cardiac insufficiency. Thus, when different treatments requiring oral taking in solid form are combined, it is important for the animal to readily accept the taking of multiple oral forms.

As regards aldosterone antagonists, patent medicines are found based on spironolactone which have to be used in combination with a standard therapy (optionally including a diuretic treatment) for the treatment of congestive cardiac insufficiency due to valve regurgitation in dogs. Spironolactone and active metabolites thereof are specific aldosterone antagonists which competitively attach to the mineralocorticoid receptors located in the kidneys, heart and blood vessels. In the kidneys, spironolactone inhibits retention of sodium induced by aldosterone, thus resulting in an increase in the excretion of sodium and consequently of water and in the retention of potassium. These renal effects result in a decrease in the extracellular volume and consequently in a reduction in the cardiac preload and in the pressure of the left atrium. This results in an improvement in the cardiac function. In the cardiovascular system, spironolactone prevents the harmful effects of aldosterone. Although its precise mechanism of action is not clearly defined, aldosterone promotes myocardial fibrosis, myocardial and vascular remodeling and endothelial dysfunction. Experimental models on dogs have shown that a long-term therapy with an aldosterone antagonist prevents the progressive dysfunction of the left ventricle and alleviates the remodeling of the left ventricle in dogs exhibiting chronic cardiac insufficiency. A clinical study has demonstrated a reduction in the deterioration in the state of the animal affected by a cardiovascular disease in dogs treated with spironolactone in addition to a standard therapy (ACEI), in comparison with the dogs treated solely with a standard therapy. Used in combination with ACEIs, spironolactone can neutralize the effect of aldosterone escape. The simultaneous taking of foods considerably increases the bioavailability of spironolactone. After an oral administration of 2 to 4 mg/kg, the absorption increases linearly with the amount administered. After multiple oral administrations of 2 mg of spironolactone per kg for 10 consecutive days, no accumulation is observed. The distribution takes place mainly towards the gastrointestinal tract, the kidneys, the liver and the adrenal glands. The spironolactone is rapidly and completely metabolized by the liver to give 7α-thiomethylspironolactone and canrenone, the main active metabolites in dogs. The Cₘₐₓ values are achieved after 2 and 4 hours respectively for the primary metabolites, 7α-thiomethylspironolactone and canrenone. A state of equilibrium is reached after 2 days. The spironolactone is mainly excreted in the form of metabolites. In dogs, 70% of the dose administered is found in the feces and 20% in the urine. It is thus again apparent that, when different treatments requiring oral taking in solid form are combined, it is important for the animal to readily accept the taking of multiple oral forms.

A first subject matter of the present invention is the use of a coating composition comprising an appropriate amount of an appetizing material capable of being applied by a film-coating process (consisting in applying a fine layer of the coating composition at the surface) for the coating by film-coating of a solid veterinary composition for oral administration indicated in the case of cardiac insufficiency, that is to say recommended and prescribed by the veterinarian for the and/or treatment of cardiac insufficiency in animals, in particular in domestic carnivores and more particularly still in the dog or cat.

The solid veterinary pharmaceutical composition for oral administration according to the invention is a medicament for veterinary use intended for the treatment of a pathological condition related to cardiac insufficiency of an animal, in particular a dog or a cat.

Thus, the present invention relates to the use of a coating composition capable of being applied by a film-coating process to a solid veterinary pharmaceutical composition for oral administration, characterized in that:

(i) the coating composition comprises:
a material appetizing for the target animal, which is provided in the pulverulent form, advantageously chosen from substances of animal or plant origin, directly brought to a powder after treatment, such as drying or dehydration, milling or grading, but also after conversion with the addition of other components in order to promote preservation, for example. The substances of choice which have a high appetence arousability for the target species, in particular domestic carnivores, such as dogs and cats, include, without implied limitation, beer yeast, meat meals, fish meals, powdered cheeses or milk derivatives, liver powder and their mixture;
at least one binder chosen from polyvinyl alcohol polymers, polyvinylpyrrolidone polymers, copolymers of vinylpyrrolidone and of vinyl acetate, polyvinyl acetate phthalate, celluloses and their derivatives, alginic acid and its salts, zein, hyaluronic acid, pectins, gum arabic, gum tragacanth, karaya gum, xanthan gum, carrageenans, pullulan or agar polymers, chitosan or its derivatives, carbomers, acrylic acid crosslinked with polyalkenyl ethers, polycarbophils, copolymers of methyl vinyl and of maleic anhydride, nonionic polyoxyethylene/polyoxypropylene block copolymers, monosaccharides, disaccharides and polysaccharides, polyols or the mixture of at least two of these binders; it being understood that, when the binder comprises one or more mono-, di- and/or polysaccharides, the percentage by weight of said mono-, di- or polysaccharides represents 50% or less, preferably 25% or less, of the total weight of binders; and
a solvent chosen from water, methanol, ethanol, isopropanol, propylene glycol, glycerol or their mixture;

(ii) it comprises between 30 and 90% inclusive by weight of appetizing material, with respect to the total weight of the mixture composed of the binder and the appetizing material; and

(iii) said solid veterinary composition for oral administration is indicated for the prevention and/or treatment of cardiac insufficiency in animals.

The appetizing material for the target animal is advantageously provided in the pulverulent form, that is to say that it is composed of small particles; this form contributes significantly to maintaining the nature appetizing for the animal of the appetizing material, even after the coating by film-coating of said material over a solid veterinary pharmaceutical composition for oral administration. This is because the pulverulent form exhibits, in comparison with the liquid forms in particular, the advantage of promoting the restoration of particles and thus of maximizing the surface areas for exchanges with the outside after application of the coating composition to the solid veterinary pharmaceutical composition to be coated.

The attractiveness for the animal of the coated solid veterinary pharmaceutical composition will depend more particularly on the high content of appetizing material in the coating composition, on its location at the surface of the solid veterinary pharmaceutical composition and on a surface area for exchange which is as large as possible by virtue of the presence of small particles. The mean diameter of the particles constituting the material appetizing for the target animal, which is provided in the pulverulent form, will advantageously be less than 500 μm, preferably less than 400 μm and more preferably still between 50 and 100 μm.

Use may be made, as appetizing material, of any foodstuff which, for a target animal, brings about an attraction, it being possible for said attraction to be evaluated by an appetence test.

The solvent is used to dissolve or suspend the binder and the pulverulent appetizing material; its content is chosen by a person skilled in the art according to the nature of said binder and of said pulverulent appetizing material. The ratio of weight of solvent to the total weight of the mixture formed by the binder and the appetizing material is preferably at least greater than 2 and less than 6 and more preferably still between 2 and 4.

The choice of the binder used in the coating composition must make the appetizing material available in order for it to exert as well as possible its function on the animal. Care thus needs to be taken to limit the phenomenon of encapsulation of the appetizing material in the pulverulent form in crystals which the binder might form during the surface drying of the coating composition after its application to the solid veterinary pharmaceutical composition.

The term “celluloses and their derivatives” is understood to mean in particular microcrystalline cellulose or ethers or esters of alkylcelluloses, such as methylcellulose, ethylcellulose, hydroxypropylcellulose, hydroxypropylmethylcellulose, cellulose acetate phthalate or cellulose acetate.

The monosaccharides, disaccharides and polysaccharides can be chosen from sucrose, glucose, maltose, dextrin, xylose, ribose, galactose, levulose, lactose or invert sugar (mixture of fructose and glucose obtained by hydrolysis of sucrose).

When monosaccharides, disaccharides and polysaccharides are used as binders, they are used as a mixture with at least one binder of another chemical family (that is to say, which is neither a mono- nor a di- nor a polysaccharide) and the percentage by weight of the monosaccharides, disaccharides and polysaccharides does not exceed 50%, preferably 25%, of the total weight of the binders.

The polyols can be chosen from sorbitol, xylitol, isomalt, maltitol, mannitol, lactitol or their mixture.

Preferably, the appetizing material is liver powder, beer yeast or a mixture of liver powder and beer yeast.

Preferably, the binder used is chosen from celluloses or their mixtures preferably comprising hydroxypropylmethylcellulose and microcrystalline cellulose.

Preferably, the coating composition comprises between 40 and 90% inclusive by weight of appetizing material, with respect to the total weight of the mixture formed by the binder and the appetizing material; more preferably, it comprises between 50 and 90%, more preferably still between 60 and 90%, inclusive by weight of appetizing material, with respect to the total weight of the mixture formed by the binder and the appetizing material. More preferably still, the coating composition comprises between 60 and 80% inclusive by weight of appetizing material, with respect to the total weight of the mixture formed by the binder and the appetizing material.

Thus composed of these three ingredients: appetizing material, binder and solvent, the coating composition can be used with the exclusion of any other ingredient.

According to another alternative form of the invention, the coating composition additionally comprises one or more acceptable additives chosen, by way of example and without any limiting nature, from plasticizers, such as stearic acid and its derivatives, citric acid and its derivatives, lactic acid and its derivatives, propylene glycols, glycerol, phthalates and their derivatives, adipates and their derivatives, sebacates and their derivatives, or polyethylene glycols and their derivatives; stabilizing agents, such as antioxidants or preservatives, such as ascorbic acid and its derivatives or salts, butylated hydroxyanisole, butylated hydroxytoluene, gallic acid and its derivatives, sodium metabisulfite, potassium metabisulfite, sodium bisulfite, vitamins and their derivatives, EDTA and its salts, or parabens; fillers, such as talc, silica, silicates, microcrystalline cellulose, mica, carbonates or silicones; surfactants; colorants, such as iron oxides, soluble dyes absorbed on alumina lakes, or titanium oxide; or porogenic agents.

The invention also relates to a process for coating by film-coating which makes it possible to fix an appetizing material, preferably a pulverulent appetizing material, at the surface of a solid veterinary pharmaceutical composition for oral administration for the prevention and/or treatment of cardiac insufficiency in animals. The film-coating of the solid veterinary pharmaceutical composition for oral administration for the prevention and/or treatment of cardiac insufficiency in animals is obtained according to conventional techniques and equipment known to a person skilled in the art.

More particularly, the present invention relates to a process for the preparation of a solid veterinary pharmaceutical composition for oral administration for the prevention and/or treatment of cardiac insufficiency in animals coated with an appetizing material (hereinafter denoted appetizing composition for oral administration for the prevention and/or treatment of cardiac insufficiency in animals), characterized in that it comprises the following stages:

(1) application, to a solid veterinary pharmaceutical composition for oral administration, of a layer of a coating composition comprising at least one appetizing material for the target animal in the pulverulent form, a binder and a solvent;

(2) drying said coated composition obtained in stage (1);

(3) optionally, repetition of stages (1) and (2); and

(4) production of an appetizing composition for oral administration for the prevention and/or treatment of cardiac insufficiency in animals in which said appetizing material represents at least 5% by weight and preferably at least 15% by weight of said appetizing composition for oral administration for the prevention and/or treatment of cardiac insufficiency in animals.

According to a specific embodiment, the process according to the invention is characterized in that it comprises the following stages:

(A) introduction of a solid veterinary pharmaceutical composition for oral administration for the prevention and/or treatment of cardiac insufficiency in animals to be coated into a coating pan;

(B) spraying, using a nozzle, a coating composition comprising at least one appetizing material for the target animal in the pulverulent form, a binder and a solvent into said rotating pan in an amount such that said appetizing material represents at least 5% by weight and preferably at least 15% by weight of the solid veterinary pharmaceutical appetizing composition for oral administration for the prevention and/or treatment of cardiac insufficiency in animals.

The temperature conditions for the implementation of the process are suitable for the equipment and the ingredients used. Advantageously, the implementation of the process according to the invention is such that the temperature of the solid veterinary pharmaceutical composition for oral administration does not exceed 42° C., preferably 40° C. and even preferably 38° C.

The coating requires the use of a coating composition as defined above, which is an aqueous or organic solution or suspension of at least one binder chosen, by way of example and without a limiting nature, from polyvinyl alcohol polymers, polyvinylpyrrolidone polymers, copolymers of vinylpyrrolidone and of vinyl acetate, polyvinyl acetate phthalate, celluloses and their derivatives, such as ethers or esters of alkylcelluloses, such as methylcellulose, ethylcellulose, hydroxypropylcellulose, hydroxypropylmethylcellulose, cellulose acetate phthalate or cellulose acetate, alginic acid and its salts, zein, hyaluronic acid, pectins, gum arabic, gum tragacanth, karaya gum, xanthan gum, carrageenans, pullulan or agar polymers, chitosan or its derivatives, carbomers, acrylic acid crosslinked with polyalkenyl ethers, polycarbophils, copolymers of methyl vinyl and of maleic anhydride, nonionic polyoxyethylene/polyoxypropylene block copolymers, monosaccharides, disaccharides and polysaccharides chosen from sucrose, glucose, maltose, dextrins, xylose, ribose, galactose, levulose, lactose or invert sugar (mixture of glucose and fructose obtained by hydrolysis of sucrose), or polyols chosen from sorbitol, xylitol, isomalt, maltitol, mannitol or lactitol; or the mixture of at least two of these binders in a solvent and which comprises the appetizing material.

When monosaccharides, disaccharides and polysaccharides are used as binders, they are used as a mixture with at least one binder of another chemical family (that is to say, which is neither a mono- nor a di- nor a polysaccharide) and the percentage by weight of the monosaccharides, disaccharides and polysaccharides does not exceed 50%, preferably 25%, of the total weight of the binders.

The coating is obtained by application of the solution or suspension and evaporation of the solvent. This application can be repeated several times, so as to obtain the desired level of appetizing material.

The coating composition according to the invention is either aqueous or organic, with solvents such as methanol, ethanol, isopropanol, propylene glycol or glycerol, or also an aqueous/alcoholic solution.

The present invention relates particularly to a solid veterinary pharmaceutical appetizing composition for oral administration for the prevention and/or treatment of cardiac insufficiency in animals comprising, as active compound, pimobendan, which exhibits an unpleasant taste or smell which thus interferes with the taking of the veterinary medicament by the target animal. This active compound may be unstable, for example with regard to the appetizing material, which would interfere with the production of an appetizing medicament and the taking of the veterinary medicament by the target animal due to the impossibility of using this appetizing material.

The solid veterinary pharmaceutical composition for oral administration is, for example and without this list having a limiting nature, a tablet, including a compressed tablet, a hard gelatin capsule, a soft capsule, a pill, a lozenge, granules, a chewing gum or a pastille.

According to an alternative form of the subject matter of the invention, the solid veterinary pharmaceutical appetizing composition for oral administration for the prevention and/or treatment of cardiac insufficiency in animals comprising is a hard gelatin capsule for oral administration which comprises at least one excipient exhibiting an unpleasant taste or smell which thus interferes with the taking of the veterinary composition by the target animal and/or one excipient which is unstable, for example with regard to the appetizing material, which interferes with the production of an appetizing medicament and the taking of the veterinary composition by the target animal.

The term “excipient” is understood to mean any ingredient of said solid veterinary pharmaceutical composition for oral administration. It concerns, for example and without a limiting nature, fillers, preservatives, stabilizing agents, colorants, porogenic agents, disintegrating agents, binders, acidifying agents or flow agents, such as described in German patent DE 4001622 or PCT WO 2005/084647.

In addition, the solid veterinary pharmaceutical composition for oral administration is optionally scored. This is because it has been realized that the action of producing, on one or both main faces of the solid veterinary pharmaceutical appetizing composition for oral administration, one or more, in particular two, notches acting as score line does not in any way detract from the ease of the taking of said solid veterinary pharmaceutical appetizing composition for oral administration or of the pieces obtained after splitting along the score line or lines.

Thus, an alternative embodiment of the process according to the invention consists in using a solid veterinary pharmaceutical composition for oral administration which has a scored form which allows it, after the stage of coating by film-coating, to be able to be divided according to the requirements of the animal to be cared for.

According to another advantageous embodiment of the process according to the invention, the solid veterinary pharmaceutical composition for oral administration for the prevention and/or treatment of cardiac insufficiency in animals is separated from the coating composition comprising the appetizing material by a film described as primer film. This primer film can be obtained by an encapsulation, a coating or a film-coating of said solid veterinary pharmaceutical composition for oral administration for the prevention and/or treatment of cardiac insufficiency in animals by a film, mainly a polymeric film, prior to the coating by the coating composition comprising the appetizing material. This primer film can have the role of producing, according to the constituents chosen, an isolating barrier and of thus protecting the solid veterinary pharmaceutical composition for oral administration for the prevention and/or treatment of cardiac insufficiency in animals, in particular the active principle or principles, from any physical or chemical attack liable to be brought about by the coating composition according to the invention. The primer film can also have the role of facilitating the adhesion of the coating composition bringing the appetizing material to the surface of the solid veterinary pharmaceutical composition for oral administration. Finally, the primer film can have the role both of providing an isolating barrier at the surface of the solid veterinary pharmaceutical composition for oral administration and of facilitating the adhesion of the coating composition bringing the appetizing material to the surface of the same solid veterinary pharmaceutical composition for oral administration.

Thus, the process according to the invention can comprise an additional stage prior to stage (1) which consists in covering, that is to say in film-coating or coating, said solid veterinary pharmaceutical composition for oral administration for the prevention and/or treatment of cardiac insufficiency in animals with a primer film using a solution or suspension comprising at least one binder and at least one solvent and/or one or more additives acceptable for the desired effect; the binder, the solvent and the acceptable additives are as defined above.

Just as for the application of the coating composition according to the invention at the surface of the solid veterinary pharmaceutical composition for oral administration for the prevention and/or treatment of cardiac insufficiency in animals, the prior application of the primer film at the surface of the solid veterinary pharmaceutical composition for oral administration is carried out by conventional techniques for coating or film-coating solid pharmaceutical compositions.

Another subject matter of the present invention is an appetizing solid veterinary pharmaceutical composition for oral administration for the prevention and/or treatment of cardiac insufficiency in animals comprising pimobendan as preferred active ingredient, characterized in that it is capable of being obtained by the process for coating by film-coating according to the invention.

This appetizing solid veterinary pharmaceutical composition for oral administration for the prevention and/or treatment of cardiac insufficiency in animals is characterized in that it comprises:
a solid veterinary pharmaceutical composition for oral administration for the prevention and/or treatment of cardiac insufficiency in animals comprising at least one active principle; and
a coating composition positioned around said solid veterinary pharmaceutical composition for oral administration for the prevention and/or treatment of cardiac insufficiency in animals of an appetizing material and of a binder, said coating composition comprising between 30 and 90% inclusive by weight of appetizing material, with respect to the total weight of the coating composed of the binder and the appetizing material, said appetizing material representing at least 5% by weight and preferably at least 15% by weight of said appetizing solid veterinary pharmaceutical composition for oral administration for the prevention and/or treatment of cardiac insufficiency in animals.

The appetizing material and the binder are as defined above.

The preferred active principle of the solid veterinary pharmaceutical composition for oral administration for the prevention and/or treatment of cardiac insufficiency in animals is pimobendan.

The preferred form of the solid veterinary pharmaceutical composition for oral administration for the prevention and/or treatment of cardiac insufficiency in animals is a hard gelatin capsule.

The advantage presented by this specific form is that it leads to an appetizing solid veterinary pharmaceutical composition for oral administration for the prevention and/or treatment of cardiac insufficiency in animals is that the appetizing material is concentrated at the surface of said appetizing hard gelatin capsule for oral administration. Thus, on using a limited and economical amount of appetizing material, a very good effectiveness in taking the appetizing medicament is nevertheless obtained, as is demonstrated in the following examples.

This appetizing solid veterinary pharmaceutical composition for oral administration for the prevention and/or treatment of cardiac insufficiency in animals is particularly advantageous for the administration of pimobendan, which is poorly accepted by animals. However, it can be used to promote the administration of any other active principle of veterinary interest for the prevention and/or treatment of cardiac insufficiency in animals in so far as it facilitates the taking of the veterinary composition and it promotes the preservation of the active principle.

Generally, the appetizing solid veterinary pharmaceutical composition for oral administration for the prevention and/or treatment of cardiac insufficiency in animals comprises between 0.01 and 500 inclusive by weight, with respect to the total weight of the appetizing solid veterinary pharmaceutical composition for oral administration for the prevention and/or treatment of cardiac insufficiency in animals, of pimobendan.

Preferably, the content of active principle does not exceed 35% by weight of the weight of the appetizing solid veterinary pharmaceutical composition for oral administration for the prevention and/or treatment of cardiac insufficiency in animals.

The pimobendan can be simply distributed within the solid veterinary pharmaceutical composition for oral administration.

The pimobendan can be itself encapsulated or coated by techniques known to a person skilled in the art in order to improve its stability or to increase the masking of its smell and its taste from the olfactory or gustatory perception of the animal.

The invention also relates to the use of pimobendan in the manufacture of a solid veterinary pharmaceutical appetizing composition for oral administration for the prevention and/or treatment of cardiac insufficiency in animals according to the invention.

Preferably, the appetizing solid veterinary pharmaceutical composition for oral administration for the prevention and/or treatment of cardiac insufficiency in animals is a hard gelatin capsule.

The solid veterinary pharmaceutical appetizing composition for oral administration is preferably intended for a domestic carnivorous animal, such as a dog or a cat.

The following nonlimiting examples illustrate the invention.

### EXAMPLE 1

### Preparation of Coated Hard Gelatin Capsules According to the Invention

The following dispersion of appetizing material to be sprayed is prepared:

| | |
|---|---|
| water | 452 g |
| hydroxypropylmethylcellulose/ | 45 g |
| microcrystalline cellulose/macrogol | |
| stearate | |
| poultry liver powder | 115 g |

for encapsulating 765 g of hard gelatin capsules which weigh 130 mg and which comprise a mixture of pimobendan, anhydrous citric acid, anhydrous colloidal silica, microcrystalline cellulose, povidone and magnesium stearate.

The hard gelatin capsules are introduced into the coating pan, where they are freed from dust and brought to temperature.

The suspension is filtered beforehand through a 500 μm sieve.

The encapsulation is carried out for approximately 2 h 15 min with a flow rate increasing from 3.5 to 4.6 g/min using a nozzle with a diameter of 1.8 mm.

The spraying of the suspension of appetizing material is halted when the required amount of appetizing material is reached.

The temperature of the incoming air is kept at approximately 55° C., while checking that the temperature of the hard gelatin capsules does not exceed 38-40° C.

At the end of the coating operation, the temperature of the coated hard gelatin capsules is brought back to 25° C.

The amount of appetizing material, the poultry liver powder, deposited on the hard gelatin capsule is calculated as follows:
the weight of 50 uncoated hard gelatin capsules is 6.50 g, i.e. 130 mg for one hard gelatin capsule, and
the weight of 50 coated hard gelatin capsules is 8.38 g, i.e. 167.6 mg for one coated hard gelatin capsule.

Thus, the weight of the coating is 37.6 mg, composed of 10.53 mg (28% w/w of the weight of the coating) of binders and 27.07 mg (72% w/w of the weight of the coating) of appetizing material, i.e. 16.15% w/w of appetizing material with respect to the total weight of the coated hard gelatin capsule.

### EXAMPLE 2

### Monadic Test of Appetence and of Stability with the Coated Hard Gelatin Capsules of Example 1

a) Appetence

A test of appetence of the appetizing hard gelatin capsules for oral administration obtained in example 1 and of the uncoated medicament (gelatin capsules no. 3 with a weight of 130 mg comprising pimobendan) was carried out with thirty six adult dogs, males and females, of varied breeds in a cross test.

On the first day, 18 dogs were tested with the coated hard gelatin capsules of example 1 and 18 dogs were tested with the uncoated hard gelatin capsules. The dogs with a body weight of less than 20 kg of each of the two groups received 1 coated hard gelatin capsule or 1 uncoated hard gelatin capsule and the dogs weighing more than 20 kg of each of the two groups received 2 coated hard gelatin capsules or 2 uncoated hard gelatin capsules.

On the third day, the first group of 18 dogs was tested with the uncoated hard gelatin capsules while the second group of 18 dogs was tested with the coated hard gelatin capsules of example 1. The dogs with a body weight of less than 20 kg of each of the two groups received 1 coated hard gelatin capsule or 1 uncoated hard gelatin capsule and the dogs weighing more than 20 kg of each of the two groups received 2 coated hard gelatin capsules or 2 uncoated hard gelatin capsules.

This is a monadic test carried out over two days of testing and carried out in individual boxes for ten minutes per dog. The following were measured:
the prehension
   from the hand,
   from the ground, or
   not taken
   the consumption
   partial,
   total, or
   no consumption.

The number of individuals, over the whole of the panel and by size categories (small/medium-sized/large), is specified for each of the criteria.

The calculation of the acceptability is based on the percentage of dogs who have consumed all of the coated or uncoated hard gelatin capsules.

Prehension:

Coated Gelatin Capsules According to the Invention (Example 1)

| | | Medium-sized dogs | | |
|---|---|---|---|---|
| | Small dogs | <20 kg | >20 kg | Large dogs |
| from the hand | 12 | 7 | 3 | 9 |
| from the ground | 0 | 0 | 1* | 3* |
| not taken | 0 | 0 | 1** | 0 |

Uncoated Gelatin Capsules

| | | Medium-sized dogs | | |
|---|---|---|---|---|
| | Small dogs | <20 kg | >20 kg | Large dogs |
| from the hand | 7 | 4 | 2 | 4 |
| from the ground | 3 | 2 | 1* | 3* |
| not taken | 2 | 1 | 1/1** | 3/2** |

*1 gelatin capsule is taken from the hand and the second from the ground
**1 gelatin capsule is taken from the hand or from the ground and the second is not taken

Consumption: Only the animals who have taken the product are considered.

Coated Gelatin Capsules According to the Invention (Example 1)

| | | Medium-sized dogs | | |
|---|---|---|---|---|
| | Small dogs | <20 kg | >20 kg | Large dogs |
| partial | 0 | 0 | 1 | 0 |
| total | 12 | 7 | 3 | 12 |
| no consumption | 0 | 0 | 0 | 0 |

Uncoated Gelatin Capsules

| | | Medium-sized dogs | | |
|---|---|---|---|---|
| | Small dogs | <20 kg | >20 kg | Large dogs |
| partial | 1*** | 0 | 2*** | 2*** |
| total | 2/3**** | 2/2**** | 1**** | 3/1**** |
| no consumption | 4 | 2 | 0 | 1 |

***There is reason to mention the behavior of the animals with this type of support: in general, the dogs took the support into the mouth. In view of the adhesiveness of the gelatin capsule in the oral cavity in the presence of the saliva due to its gelatin composition, the animals attempted to spit out the gelatin capsule or capsules, which resulted in them becoming pierced, which had the effect of dispersing a portion of the contents of the latter on the ground when it was not the gelatin capsule which was rejected directly, all the more so as the animal shook its head, the mouth downward. In this scenario, a “partial consumption” value was assigned to these cases.
****As above, the dogs rejected the hard gelatin capsule or a portion of its contents. In this scenario, when some animals licked all or part of the medicinal composition from the ground, a “total consumption” value was assigned to these cases.

For the coated hard gelatin capsules according to the invention, the acceptability was 97.22% and the total consumption was 94.44%, whereas the acceptability of the uncoated hard gelatin capsules was 77.22% and their total consumption was 38.88%.

The simultaneous taking of several coated hard gelatin capsules according to the invention is 94.11% and their total consumption is 88.23%, whereas the simultaneous taking of the uncoated hard gelatin capsules was 58.82% and their maximum total consumption was 29.41%, making the approximation which consists in regarding the animals who licked the medicinal composition from the ground as having consumed all of the dose administered.

### EXAMPLE 3

### Preparation of Coated Hard Gelatin Capsules Film-Coated Beforehand According to the Invention

1—Film-Coating with an Isolating Primer Film

The following dispersion to be sprayed, in order to form an isolating primer film, is prepared:

| | |
|---|---|
| water | 140.25 g |
| hydroxypropylmethylcellulose/ | 19.125 g |
| microcrystalline cellulose/stearic | |
| acid | |

for encapsulating 765 g of hard gelatin capsules weighing 130 mg (with the composition: pimobendan/anhydrous citric acid/anhydrous colloidal silica/micro-crystalline cellulose/povidone/magnesium stearate).

The hard gelatin capsules are introduced into the coating pan, where they are freed from dust and brought to temperature.

The coating is carried out for approximately 2 h with a flow rate increasing from 3.1 to 4.56 g/min using a nozzle with a diameter of 1 mm.

The temperature of the incoming air is kept at approximately 50° C., while checking that the temperature of the gelatin capsules does not exceed 40-42° C.

At the end of the film-coating operation, the temperature of the hard gelatin capsules film-coated with the isolating primer film is brought back to 25° C.

2—Coating with the Appetizing Material

The following dispersion of appetizing material to be sprayed is prepared:

| | |
|---|---|
| water | 570.0 g |
| hydroxypropylmethylcellulose/ | 56.75 g |
| microcrystalline cellulose/macrogol | |
| stearate | |
| poultry liver powder | 145.0 g |

for coating the film-coated hard gelatin capsules obtained in the above stage 1.

The suspension is filtered beforehand through a 500 μm sieve.

The coating is carried out for approximately 5 h with a flow rate increasing from 3.5 to 4.6 g/min using a nozzle with a diameter of 1.8 mm.

The temperature of the incoming air is kept at approximately 50-55° C., while checking that the temperature of the compressed tablets does not exceed 38-40° C.

The spraying of the suspension of appetizing material is halted when the required amount of appetizing material is reached.

At the end of the coating operation, the temperature of the hard gelatin capsules coated with appetizing material is brought back to 25° C.

The amount of appetizing material, the poultry liver powder, deposited on the hard gelatin capsule is calculated as follows:
the weight of 50 hard gelatin capsules is 6.5 g, i.e. 130 mg for one hard gelatin capsule,
the weight of 50 film-coated hard gelatin capsules is 6.65 g, i.e. 133 mg for one hard gelatin capsule, and
the weight of 50 coated hard gelatin capsules is 8.42 g, i.e. 168.4 mg for one coated hard gelatin capsule.

Thus, the weight of the coating is 35.4 mg, composed of 9.97 mg (28.18% w/w of the weight of the coating) of binder and 25.44 mg (71.87% w/w of the weight of the coating) of appetizing material, i.e. 15.10% w/w of appetizing material with respect to the total weight of the coated hard gelatin capsule.

1. A method of coating a solid veterinary pharmaceutical composition with a coating composition, the method comprising:
film-coating the solid veterinary pharmaceutical composition comprising at least one compound for treating cardiac insufficiency, with the coating composition,
wherein the coating composition comprises:
between 30 and 90% inclusive by weight of an appetizing material for a target animal, in pulverulent form, with respect to a total weight of binder and the appetizing material;
at least one binder selected from the group consisting of a polyvinyl alcohol polymer, a polyvinylpyrrolidone polymer, a copolymer of vinylpyrrolidone, a copolymer of vinyl acetate, polyvinyl acetate phthalate, a cellulose, a cellulose derivative, alginic acid, an alginic acid salt, zein, hyaluronic acid, a pectin, gum arabic, gum tragacanth, karaya gum, xanthan gum, a carrageenan, a pullulan polymer, an agar polymer, chitosan, a chitosan derivative, a carbomer, acrylic acid crosslinked with a polyalkenyl ether, a polycarbophil, a copolymer of methyl vinyl and of maleic anhydride, a nonionic polyoxyethylene/polyoxy-propylene block copolymer, a monosaccharide, a disaccharide, a polysaccharide, and a polyol; it being understood that, when the binder comprises one or more mono-, di- and/or polysaccharides, a percentage by weight of the mono-, di- or polysaccharides represents 50% or less, of a total weight of binders;
at least one solvent selected from the group consisting of water, methanol, ethanol, isopropanol, propylene glycol, and glycerol;
and
wherein the solid veterinary pharmaceutical composition
is suitable for oral administration, and
is indicated for preventing and/or treating cardiac insufficiency in an animal.

2. The method of claim 1, wherein the solid veterinary pharmaceutical composition comprises pimobendan.

3. The method of claim 1, wherein the appetizing material is at least one selected from beer yeast, a meat meal, a fish meal, a powdered cheese, a milk derivative, and liver powder.

4. The method of claim 1, wherein the appetizing material comprises particles having a mean diameter of less than 500 μm.

5. The method of claim 1, wherein the coating composition further comprises:
at least one additive selected from a plasticizer; a stabilizing agent; a preservative; a filler; a surfactant; a colorant, and a porogenic agent.

6. The method of claim 1, wherein the solid veterinary pharmaceutical composition is suitable for a domestic carnivorous animal.

7. The method of claim 6, wherein the domestic carnivorous animal is a dog or a cat.

8. A process for preparing a solid veterinary pharmaceutical composition for oral administration coated with at least one appetizing material, the process comprising:
(1) applying to the solid veterinary pharmaceutical composition a layer of a coating composition comprising the at least one appetizing material for a target animal in a pulverulent form, a binder, and a solvent, to obtain a coated composition;
(2) drying the coated composition obtained in (1); and
(3) optionally, repeating (1) and (2);
to produce a solid veterinary pharmaceutical appetizing composition suitable for oral administration for preventing and/or treating cardiac insufficiency in an animal,
wherein the appetizing material represents at least 5% by weight of the solid veterinary pharmaceutical appetizing composition
and wherein the solid veterinary pharmaceutical composition:
prevents and/or treats cardiac insufficiency in an animal; and
comprises at least one compound for treating cardiac insufficiency.

9. The process of claim 8, wherein the solid veterinary composition comprises pimobendan.

10. The process of claim 8, wherein the solid veterinary pharmaceutical composition is a tablet, a compressed tablet, a hard gelatin capsule, a soft capsule, a pill, a lozenge, granules, a chewing gum, or a pastille.

11. The process of claim 10, wherein the solid veterinary pharmaceutical composition is a hard gelatin capsule.

12. The process of claim 8, wherein the solid veterinary pharmaceutical composition is scored.

13. The process of claim 8, further comprising, prior to (1),
(0) coating the solid veterinary pharmaceutical composition with a primer film from a solution or suspension comprising at least one binder and at least one solvent and/or acceptable additive.

14. A solid veterinary pharmaceutical appetizing composition, comprising:
a solid veterinary pharmaceutical composition comprising at least one active principle comprising a compound for treating cardiac insufficiency; and
a coating positioned around the solid veterinary pharmaceutical appetizing composition, which comprises:
an appetizing material in pulverulent form; and
at least one binder selected from the group consisting of a polyvinyl alcohol polymer, a polyvinylpyrrolidone polymer, a copolymer of vinylpyrrolidone, a copolymer of vinyl acetate, polyvinyl acetate phthalate, a cellulose, a cellulose derivative, alginic acid, an alginic acid salt, zein, hyaluronic acid, a pectin, gum arabic, gum tragacanth, karaya gum, xanthan gum, a carrageenan, a pullulan polymer, an agar polymer, chitosan, a chitosan derivative, a carbomer, acrylic acid crosslinked with a polyalkenyl ether, a polycarbophil, a copolymer of methyl vinyl and of maleic anyhydride, a nonionic polyoxyethylene/polyoxy-propylene block copolymer, a monosaccharide, a disaccharide, a polysaccharide, and a polyol; it being understood that, when the binder comprises one or more mono-, di- and/or polysaccharides, a percentage by weight of the mono-, di- or polysaccharides represents 50% or less, of a total weight of binders,
wherein the coating comprising between 30 and 90% inclusive by weight of an appetizing material, with respect to a total weight of the coating,
an appetizing material represents representing at least 5% by weight of the solid veterinary pharmaceutical appetizing composition for oral administration,
the solid veterinary pharmaceutical appetizing composition is for oral administration and prevents and/or treats cardiac insufficiency in an animal.

15. The composition of claim 14, wherein the active principle of the solid veterinary pharmaceutical composition is pimobendan.

16. The composition of claim 14, suitable for a cat or a dog.

17. A method of manufacturing a solid veterinary pharmaceutical composition comprising pimobendan, the method comprising
combining pimobendan with contents within the solid veterinary pharmaceutical appetizing composition of claim 14.

18. The method of claim 2, wherein the appetizing material is at least one selected from beer yeast, a meat meal, a fish meal, a powdered cheese, a milk derivative, and liver powder.

19. The method of claim 1, wherein the appetizing material comprises particles having a mean diameter of less than 400 μm.

20. The method of claim 1, wherein the appetizing material comprises particles having a mean diameter of between 50 and 100 μm.
